(19) United States
(12) Patent Application Publication
(10) Pub. No.: US 2024/0032999 A1
(43) Pub. Date: Feb. 1, 2024

(54) METHOD AND APPARATUS FOR NON-INVASIVE IMAGE-OBSERVING DENSITY OF INTRA-EPIDERMAL NERVE FIBER OF HUMAN SKIN

(71) Applicant: NATIONAL TAIWAN UNIVERSITY, Taipei City (TW)

(72) Inventors: CHI-KUANG SUN, Taipei City (TW); PEI-JHE WU, Taipei City (TW); HSIAO-CHIEH TSENG, Taipei City (TW)

(73) Assignee: NATIONAL TAIWAN UNIVERSITY, Taipei City (TW)

(21) Appl. No.: 17/815,571

(22) Filed: Jul. 28, 2022

(51) Int. Cl. A61B 18/20; A61N 5/06

(52) U.S. Cl. CPC A61B 18/203; A61N 5/0616; A61B 2018/00452

(57) ABSTRACT

The present invention relates to a method and apparatus for non-invasive image-observing the density of an intra-epidermal nerve fiber of human skin, in which the method includes: providing a nonlinear optical microscopy device for capturing an intra-epidermal nerve fiber structural image of an acquisition area of a to-be-tested human skin to observe continuous signals of intra-epidermal nerve fiber images, wherein the nonlinear optical microscopy device includes: a laser light source for emitting laser light with a pulsed laser, and an image processing member for processing image signals; focusing the laser light on the intra-epidermal nerve fiber to obtain nerve signals of the intra-epidermal nerve fiber that have a length of at least three points of the intra-epidermal nerve fiber, and constitute a plurality of nerve fibers; and calculating the total number of nerve fiber signals of the to-be-tested human skin, and dividing it by the total area of captured images to obtain the density of the to-be-tested human body; and evaluating and determining whether the human suffers from related neuropathy. such as peripheral neuropathy.

### FIELD OF THE INVENTION

The present invention relates to a method and an apparatus for non-invasive image-observing the density of an intra-epidermal nerve fiber in human skin, in which the method includes providing a nonlinear optical microscopy device for capturing an intra-epidermal nerve fiber structural image of an image-capturing area of a to-be tested human skin to observe continuous signals of the intra-epidermal nerve fiber structural image, and calculating the density (or density value) of the intra-epidermal nerve fiber of the to-be-tested human skin through an image processing member, evaluating the level of damages to the intra-epidermal nerve fiber of the to-be-tested human body, and determining whether the human body suffers from related neuropathy, such as peripheral neuropathy.

### BACKGROUND OF THE INVENTION

At present, the approach adopted by hospital neurology departments to observe nerves is to remove skin tissues from a patient by using a punch biopsy. It is an invasive method that not only causes discomfort to human body, but occasionally causes accidental injuries. In addition, when skin tissue images are observed for diagnosis, it requires special chemical immunostaining techniques to complete the observation. The medical procedure is complicated, and its accuracy is yet to be improved.

Accordingly, a non-invasive and harmless method for observing signals of an intra-epidermal nerve fiber structural image to obtain the density of the intra-epidermal nerve fiber in the epidermis of the human skin without requiring special immunohistochemistry staining technology to provide rapid and accurate diagnoses and necessary correction is expected by the medical field. Furthermore, it is a breakthrough if the density of the peripheral intra-epidermal nerve fiber can be calculated through observing the peripheral intra-epidermal nerve fiber in the epidermis of human skin to determine if the human is suffering from peripheral neuropathy.

### SUMMARY OF THE INVENTION

The present invention aims to provide a method and apparatus for non-invasive image-observing the density of an intra-epidermal nerve fiber of human skin, by using the non-invasive harmonic generation microscopy, images of the density of the intra-epidermal nerve fiber of human skin can be observed to evaluate the level of damages to the intra-epidermal nerve fiber of the to-be-tested human body and to determine whether the human body suffers from related neuropathy, such as peripheral neuropathy, in which the peripheral intra-epidermal nerve fiber is an unmyelinated fiber in the epidermis of the human skin.

Another object of the present invention is to provide a method and apparatus for non-invasive image-observing the density (density value) of the entire segment or one segment of an intra-epidermal nerve fiber in the epidermis of the human skin, which require no process of using special chemical immunostaining technology to obtain the density of the intra-epidermal nerve fiber in human skin, thereby quickly and accurately determining whether the human body suffers from peripheral neuropathy.

A method for non-invasive image-observing the density of an intra-epidermal nerve fiber of human skin is proposed, which comprises: providing a nonlinear optical microscopy device for capturing an intra-epidermal nerve fiber structural image of an acquisition area of a to-be-tested human skin to observe continuous signals of the intra-epidermal nerve fiber structural image, wherein the continuous signals of the intra-epidermal nerve fiber structural image include a string-like or a claw-like structural signal of a peripheral intra-epidermal nerve fiber formed by crossing through a junctional layer of the human skin epidermis and dermis and extending into the epidermis, and a string-like or a claw-like structural signal of the peripheral intra-epidermal nerve fiber only located in the epidermis, wherein the peripheral intra-epidermal nerve fiber is an unmyelinated fiber in the epidermis of the human skin, and wherein the nonlinear optical microscopy device includes: a laser source for emitting laser light with a pulsed laser, and an image processing member for processing image signals;
focusing the laser light on the intra-epidermal nerve fiber of the acquisition area of the to-be-tested human skin by using the image processing member to obtain continuous signals of the intra-epidermal nerve fiber structural image with a length having at least three points and continuous, wherein the continuous signals of the intra-epidermal nerve fiber structural image are third harmonic generation nonlinear optical signals generated after being excited by the laser light, the size of the signal point of each of the continuous signals of the intra-epidermal nerve fiber structural image is 200 nm or more, and the linear distance from one signal point to another signal point in a three-dimensional space ranges from 0 to 7.5 μm, and wherein the continuous signals connected together by at least three points constitute a string-like intra-epidermal nerve fiber structural signal segment, and the string-like intra-epidermal nerve fiber structural signal segment includes signal segments only existing in the epidermis, and the other signal segments extendedly crossing through the junctional layer of the skin epidermis and dermis to exist in the epidermis, and the shortest distance between the string-like intra-epidermal never fiber structural signal segments and the junctional layer of the epidermis and dermis is less than 20 μm, thereby constituting a plurality of nerve fibers;
calculating a total number of the plurality of nerve fibers of the total intra-epidermal nerve fiber in the acquisition area of the to-be-tested human skin and dividing it by a total acquisition area (mm²) of the captured images to obtain a density of the intra-epidermal nerve fiber of the to-be-tested human skin; and evaluating levels of damage to the intra-epidermal nerve fiber of the to-be-tested human skin, and determining whether related neuropathy such as peripheral neuropathy is suffered, that is, the more severe the intra-epidermal nerve fiber is damaged, the lower the density is.

An apparatus for non-invasive image-observing the density of an intra-epidermal nerve fiber of human skin is submitted, which comprises: a nonlinear optical microscopy device for capturing an intra-epidermal nerve fiber structural image of an acquisition area of a to-be-tested human skin to observe continuous signals of the intra-epidermal nerve fiber structural image, wherein the continuous signals of the intra-epidermal nerve fiber structural image include a string-like or a claw-like structural signal of a peripheral intra-epidermal nerve fiber formed by crossing through a junctional layer of the human skin epidermis and dermis and extending into the epidermis, and a string-like or a claw-like structural signal of the peripheral intra-epidermal nerve fiber only located in the epidermis, wherein the peripheral intra-epidermal nerve fiber is an unmyelinated fiber in the epidermis of the human skin, and wherein the nonlinear optical microscopy device includes: a laser source for emitting laser light with a pulsed laser, and an image processing member for processing image signals, in which the laser light is focused on the intra-epidermal nerve fiber of the acquisition area of the to-be-tested human skin by using the image processing member to obtain continuous signals of the intra-epidermal nerve fiber structural image with a length having at least three points and continuous, wherein the continuous signals of the intra-epidermal nerve fiber structural image are third harmonic generation nonlinear optical signals generated after being excited by the laser light, the size of the signal point of each of the continuous signals of the intra-epidermal nerve fiber structural image is 200 nm or more, and the linear distance from one signal point to another signal point in a three-dimensional space ranges from 0 to 7.5 μm, and wherein the continuous signals connected together by at least three points constitute a string-like intra-epidermal nerve fiber structural signal segment, and the string-like intra-epidermal nerve fiber structural signal segment includes signal segments only existing in the epidermis, and the other signal segments extendedly crossing through the junctional layer of the skin epidermis and dermis to exist in the epidermis, and the shortest distance between the string-like intra-epidermal never fiber structural signal segments and the junctional layer of the epidermis and dermis is less than 20 μm, thereby constituting a plurality of nerve fibers;
a calculation element for calculating a total number of the plurality of nerve fibers of the total intra-epidermal nerve fiber in the acquisition area of the to-be-tested human skin and dividing it by a total acquisition area (mm²) of the captured images to obtain a density of the intra-epidermal nerve fiber of the to-be-tested human skin; and
an evaluation and judging element for evaluating levels of damage to the intra-epidermal nerve fiber of the to-be-tested human skin, and determining whether related neuropathy such as peripheral neuropathy is suffered, that is, the more severe the intra-epidermal nerve fiber is damaged, the lower the density is.

### BRIEF DESCRIPTION OF THE DRAWINGS

FIG. 1 shows a flow chart of a method for non-invasive image-observing the density of an intra-epidermal nerve fiber of human skin according to the present invention.

FIG. 2 shows a block diagram of an apparatus for non-invasive image-observing the density of an intra-epidermal nerve fiber of human skin according to the present invention.

### DETAILED DESCRIPTION OF THE INVENTION

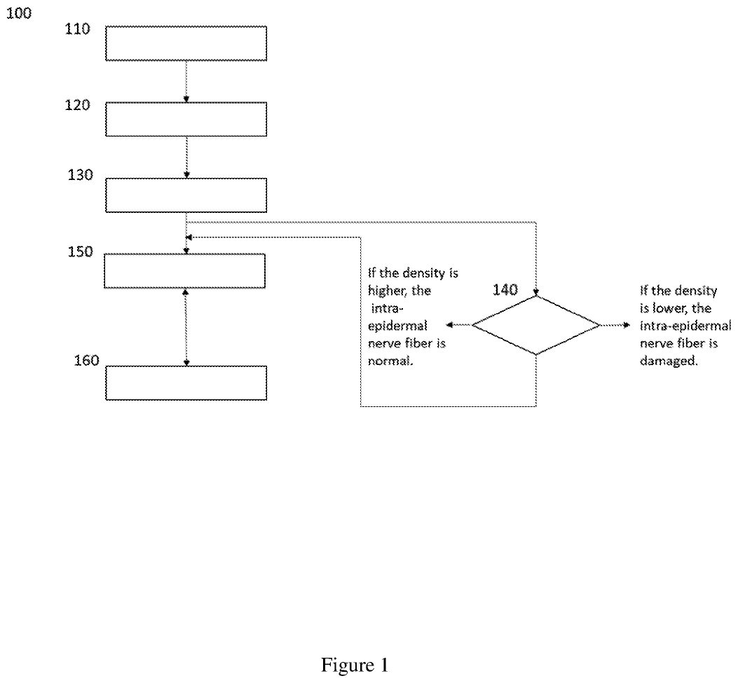

As shown in FIG. 1, a method for non-invasive image-observing the density of an intra-epidermal nerve fiber of human skin 100 according to the present invention comprises:
providing a nonlinear optical microscopy device for capturing an intra-epidermal nerve fiber structural image of an acquisition area of a to-be-tested human skin to observe continuous signals of the intra-epidermal nerve fiber structural image 110, wherein the continuous signals of the intra-epidermal nerve fiber structural image include a string-like or a claw-like structural signal of a peripheral intra-epidermal nerve fiber formed by crossing through a junctional layer of the human skin epidermis and dermis and extending into the epidermis, and a string-like or a claw-like structural signal of the peripheral intra-epidermal nerve fiber only located in the epidermis, wherein the peripheral intra-epidermal nerve fiber is an unmyelinated fiber in the epidermis of the human skin, and wherein the nonlinear optical microscopy device includes: a laser source for emitting laser light with a pulsed laser, and an image processing member for processing image signals;
focusing the laser light on the intra-epidermal nerve fiber of the acquisition area of the to-be-tested human skin by using the image processing member to obtain continuous signals of the intra-epidermal nerve fiber structural image with a length having at least three points and continuous 120, wherein the continuous signals of the intra-epidermal nerve fiber structural image are third harmonic generation nonlinear optical signals generated after being excited by the laser light, the size of the signal point of each of the continuous signals of the intra-epidermal nerve fiber structural image is 200 nm or more, and the linear distance from one signal point to another signal point in a three-dimensional space ranges from 0 to 7.5 μm, and wherein the continuous signals connected together by at least three points constitute a string-like intra-epidermal nerve fiber structural signal segment, and the string-like intra-epidermal nerve fiber structural signal segment includes signal segments only existing in the epidermis, and the other signal segments extendedly crossing through the junctional layer of the skin epidermis and dermis to exist in the epidermis, and the shortest distance between the string-like intra-epidermal never fiber structural signal segments and the junctional layer of the epidermis and dermis is less than 20 μm, thereby constituting a plurality of nerve fibers;
calculating a total number of the plurality of nerve fibers of the total intra-epidermal nerve fiber in the acquisition area of the to-be-tested human skin and dividing it by a total acquisition area (mm²) of the captured images to obtain a density of the intra-epidermal nerve fiber of the to-be-tested human skin 130; and
evaluating levels of damage to the intra-epidermal nerve fiber of the to-be-tested human skin, and determining whether related neuropathy such as peripheral neuropathy is suffered, that is, the more severe the intra-epidermal nerve fiber is damaged, the lower the density is 140.

In the method for non-invasive image-observing the density of an intra-epidermal nerve fiber of human skin of 100 of the invention, a normal value of the density of the intra-epidermal nerve fiber is from 50 to 60 (the number of the nerve fibers/mm²), and wherein the intra-epidermal nerve fiber is a C nerve fiber or A-delta nerve fiber, having a point-like structure.

In the method for non-invasive image-observing the density of an intra-epidermal nerve fiber of human skin 100 of the invention, the non-linear optical microscopy device is a third harmonic generation microscopy device (THG), a second harmonic generation microscopy device (SHG) plus a third harmonic generation microscopy device (THG), or a combination thereof, wherein the specification of the third harmonic generation microscopy device (THG) is NA≥0.75, the central wavelength: 1065-1450 nm, pulse width: <10 ps, and reverse collection of optical signals.

According to the method for non-invasive image-observing the density of an intra-epidermal nerve fiber of human skin 100 of the invention, wherein each nerve fiber extends along the same direction or a plurality of directions; or each nerve fiber is continuous in the same epidermis with no extension or is continuous in different skin layers with extensions; or each nerve fiber extends in different skin layers, which is a point-like signal and a point-like signal extension, or a point-like signal and a linear signal extension; or each nerve fiber is not arranged as a circle to eliminate cell signals in the skin.

In the method for non-invasive image-observing the density of an intra-epidermal nerve fiber of human skin 100 of the invention, the nonlinear optical microscopy device is a second harmonic generation microscopy device (SHG) plus a third harmonic generation microscopy device (THG) for observing the intra-epidermal nerve fiber structural image, and if the intra-epidermal nerve fiber extends in the horizontal direction (perpendicular to the forward direction of the laser light), and as is known through numerical simulation, it is used for generating a high-strength third harmonic generation, so that the intra-epidermal nerve fiber signal is observed in the same skin layer as linear and continuous; and if the intra-epidermal nerve fiber extends in the vertical direction (parallel to the forward direction of the laser light), it is known through numerical simulation, that if it is a simple cylindrical thin line, the intensity of the third harmonic generation is extremely small, it must conform to the structure of the varicosity to generate high-strength third harmonic generation, so that the intra-epidermal nerve fiber signal is observed in different skin layers as a point-like and point-like extension, or a point-like and linear extension.

Further, according to the invention, the method for non-invasive image-observing the density of an intra-epidermal nerve fiber of human skin 100 further comprises a displaying step for displaying the image and/or the density value of each nerve fiber 150.

Still further, method for non-invasive image-observing the density of an intra-epidermal nerve fiber of human skin 100 according to the invention, a correction step 160 is provided as follow. Wherein when observing the intra-epidermal nerve fiber image, the number of cells in a basal layer of the epidermis capable of reflecting visibility of the image is further observed, wherein the number of cells in the basal layer is used to correct the density of the entire segment of the intra-epidermal nerve fiber and the density of the intra-epidermal nerve fiber.

According to the method of the invention, examples of the observed and calculated density are given below.

### Example 1

For a single piece of picture (image) at a size of 0.5×0.5 m², taken by the non-invasive optical microscopy device of the invention, it requires a total size of at least greater than 1 mm² for capturing so as to calculate the density of the intra-epidermal nerve fiber in the skin and thus it needs at least four pictures.

Assuming 1 mm² of the total size is taken with pictures Nos. 1, 2, 3 and 4:
Picture 1, the calculated number of the nerve fiber is 12;
Picture 2, the calculated number of the nerve fiber is 15;
Picture 3, the calculated number of the nerve fiber is 18; and
Picture 4, the calculated number of the nerve fiber is 10.

The density of the intra-epidermal nerve fibers is 12+15+18+55=55 fibers/mm².

### Example 2

Assuming a total size of greater than 1 mm² is taken with pictures 1, 2, 3, 4, 5 and 6:
Picture 1, the calculated number of the nerve fiber is 12;
Picture 2, the calculated number of the nerve fiber is 15;
Picture 3, the calculated number of the nerve fiber is 18; and
Picture 4, the calculated number of the nerve fiber is 10.
Picture 5, the calculated number of the nerve fiber is 10.
Picture 6, the calculated number of the nerve fiber is 11.

The total number of the nerve fibers is 12+15+18+10+10+11=76 fibers, which are divided by the total size of 0.5×0.5×6=1.5 mm².

Then, the density of the intra-epidermal nerve fiber of 76/1.5=50.66 fibers/mm² is obtained.

Via a test of the density of a to-be-tested people suffering the peripherals neuropathy, the density is 26.35 fibers/mm², while the density of a normal people under test is 55.26 fibers/mm². Therefore, the lower the density of the intra-epidermal nerve fiber is, the severe the intra-epidermal is damaged.

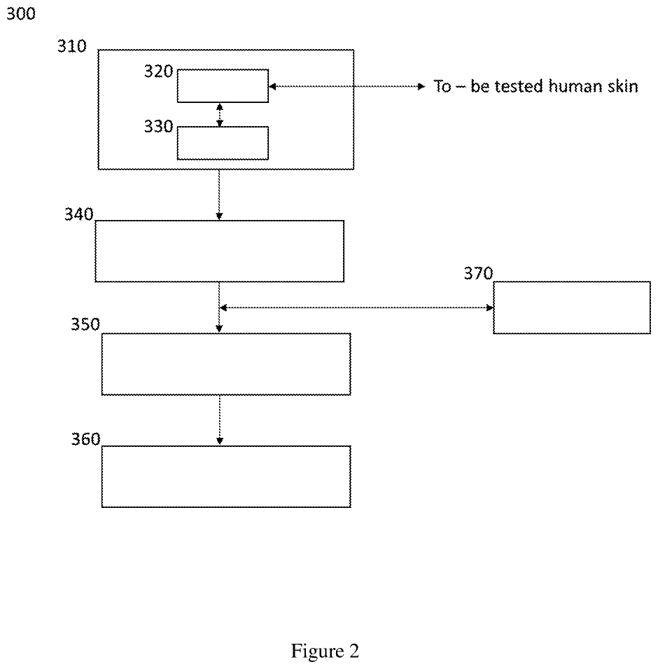

As shown in FIG. 2, an apparatus for non-invasive image-observing the density of an intra-epidermal nerve fiber of human skin 300 according to the invention comprises:
a nonlinear optical microscopy device 310 for capturing an intra-epidermal nerve fiber structural image of an acquisition area of a to-be-tested human skin to observe continuous signals of the intra-epidermal nerve fiber structural image, wherein the continuous signals of the intra-epidermal nerve fiber structural image include a string-like or a claw-like structural signal of a peripheral intra-epidermal nerve fiber formed by crossing through a junctional layer of the human skin epidermis and dermis and extending into the epidermis, and a string-like or a claw-like structural signal of the peripheral intra-epidermal nerve fiber only located in the epidermis, wherein the peripheral intra-epidermal nerve fiber is an unmyelinated fiber in the epidermis of the human skin, and wherein the nonlinear optical microscopy device includes: a laser source for emitting laser light with a pulsed laser 320, and an image processing member 330 for processing image signals, in which the laser light is focused on the intra-epidermal nerve fiber of the acquisition area of the to-be-tested human skin by using the image processing member to obtain continuous signals of the intra-epidermal nerve fiber structural image with a length having at least three points and continuous, wherein the continuous signals of the intra-epidermal nerve fiber structural image are third harmonic generation nonlinear optical signals generated after being excited by the laser light, the size of the signal point of each of the continuous signals of the intra-epidermal nerve fiber structural image is 200 nm or more, and the linear distance from one signal point to another signal point in a three-dimensional space ranges from 0 to 7.5 μm, and wherein the continuous signals connected together by at least three points constitute a string-like intra-epidermal nerve fiber structural signal segment, and the string-like intra-epidermal nerve fiber structural signal segment includes signal segments only existing in the epidermis, and the other signal segments extendedly crossing through the junctional layer of the skin epidermis and dermis to exist in the epidermis, and the shortest distance between the string-like intra-epidermal never fiber structural signal segments and the junctional layer of the epidermis and dermis is less than 20 μm, thereby constituting a plurality of nerve fibers;
a calculation element 340 for calculating a total number of the plurality of nerve fibers of the total intra-epidermal nerve fiber in the acquisition area of the to-be-tested human skin and dividing it by a total acquisition area (mm²) of the captured images to obtain a density of the intra-epidermal nerve fiber of the to-be-tested human skin; and
an evaluation and judging element 350 for evaluating levels of damage to the intra-epidermal nerve fiber of the to-be-tested human skin, and determining whether related neuropathy such as peripheral neuropathy is suffered, that is, the more severe the intra-epidermal nerve fiber is damaged, the lower the density is. In the invention, the apparatus for non-invasive image-observing the density of an intra-epidermal nerve fiber of human skin 300 further comprises a displaying element 360 for displaying the image and/or the density of each nerve fiber. In the invention, a normal value of the density of the intra-epidermal nerve fiber is from 50 to 60 (the number of the nerve fibers/mm²), and the intra-epidermal nerve fiber is a C nerve fiber or A-delta nerve fiber, having a point-like structure. Further, in the apparatus for non-invasive image-observing the density of an intra-epidermal nerve fiber of human skin 300 of the invention, the non-linear optical microscopy device is a third harmonic generation microscopy device (THG), a second harmonic generation microscopy device (SHG) plus a third harmonic generation microscopy device (THG), or a combination thereof, wherein the specification of the third harmonic generation microscopy device (THG) is NA≥0.75, the central wavelength: 1065-1450 nm, pulse width: <10 ps, and reverse collection of optical signals.

According to the apparatus for non-invasive image-observing the density of an intra-epidermal nerve fiber of human skin 300 of the invention, each nerve fiber extends along the same direction or a plurality of directions; or each nerve fiber is continuous in the same epidermis with no extension, or is continuous in different skin layers with extensions; or when each nerve fiber extends in different skin layers, it is a point-like signal and a point-like signal extension, or a point-like signal and a linear signal extension; or each nerve fiber is not arranged as a circle to eliminate cell signals in the skin.

In addition, in the apparatus for non-invasive image-observing the density of an intra-epidermal nerve fiber of human skin 300 of the invention, the nonlinear optical microscopy device is a second harmonic generation microscopy device (SHG) plus a third harmonic generation microscopy device (THG) for observing the intra-epidermal nerve fiber structural image, and if the intra-epidermal nerve fiber extends in the horizontal direction (perpendicular to the forward direction of the laser light), and as is known through numerical simulation, it is used for generating a high-strength third harmonic generation, so that the intra-epidermal nerve fiber signal is observed in the same skin layer as linear and continuous; and if the intra-epidermal nerve fiber extends in the vertical direction (parallel to the forward direction of the laser light), it is known through numerical simulation, that if it is a simple cylindrical thin line, the intensity of the third harmonic generation is extremely small, it must conform to the structure of the varicosity to generate high-strength third harmonic generation, so that the intra-epidermal nerve fiber signal is observed in different skin layers as a point-like and point-like extension, or a point-like and linear extension.

Still further, the apparatus for non-invasive image-observing the density of an intra-epidermal nerve fiber of human skin 300 of the invention further comprises a correction element 370. When observing the intra-epidermal nerve fiber image, the number of cells in a basal layer of the epidermis capable of reflecting visibility of the image is further observed, and wherein a correction element is further provided for correcting the density of the intra-epidermal nerve fiber, via using the number of cells in the basal layer.

The test data of calculating the density of the intra-epidermal using the apparatus of the invention is presented in the above method as submitted.

Steps Implemented in the Method and the Elements Employed in the Apparatus
100 A method for non-invasive image-observing the density of an intra-epidermal nerve fiber of human skin
110 providing a non-invasive, nonlinear optical microscopy device for capturing an intra-epidermal nerve fiber structural image of a to-be-tested human skin to observe continuous signals of intra-epidermal nerve fiber structural images
120 focusing the laser light, emitted by a laser source of the nonlinear optical microscopy device, on the intra-epidermal nerve fiber of the acquisition area of the to-be-tested human skin to obtain continuous signals of the intra-epidermal nerve fiber structural image having a length of at least three points, wherein the continuous connected together constitute a plurality of linear fibers
130 calculating a total number of the plurality of nerve fibers of the total intra-epidermal nerve fiber in the acquisition area of the to-be-tested human skin and dividing it by a total acquisition area (mm²) of the captured images to obtain a density of the intra-epidermal nerve fiber of the to-be-tested human skin; and
140 evaluating levels of damage to the intra-epidermal nerve fiber of the to-be-tested human skin, and determining whether related neuropathy,
such as peripheral neuropathy is suffered, that is, the more severe the intra-epidermal nerve fiber is damaged, the lower the density is.
150 displaying the image and/or the density of each nerve fiber
170 using the number of cells in a basal layer of the epidermis simultaneously observed by using the nonlinear optical microscopy device to correct the density (value) of the entire segment of the intra-epidermal fiber and the density of the intra-epidermal nerve fiber
300 An apparatus for non-invasive image-observing the density of an intra-epidermal nerve fiber of human skin
310 a non-linear optical microscopy device
320 a laser source
330 an image processing member
340 a calculation element
350 evaluating and judging element
360 displaying element
370 correction element

What is claimed is:

1. A method for non-invasive image-observing the density of an intra-epidermal nerve fiber of human skin, which comprises:
providing a nonlinear optical microscopy device for capturing an intra-epidermal nerve fiber structural image of an acquisition area of a to-be-tested human skin to observe continuous signals of the intra-epidermal nerve fiber structural image, wherein the continuous signals of the intra-epidermal nerve fiber structural image include a string-like or a claw-like structural signal of a peripheral intra-epidermal nerve fiber formed by crossing through a junctional layer of the human skin epidermis and dermis and extending into the epidermis, and a string-like or a claw-like structural signal of the peripheral intra-epidermal nerve fiber only located in the epidermis, wherein the peripheral intra-epidermal nerve fiber is an unmyelinated fiber in the epidermis of the human skin, and wherein the nonlinear optical microscopy device includes: a laser source for emitting laser light with a pulsed laser, and an image processing member for processing image signals;
focusing the laser light on the intra-epidermal nerve fiber of the acquisition area of the to-be-tested human skin by using the image processing member to obtain continuous signals of the intra-epidermal nerve fiber structural image with a length having at least three points and continuous, wherein the continuous signals of the intra-epidermal nerve fiber structural image are third harmonic generation nonlinear optical signals generated after being excited by the laser light, the size of the signal point of each of the continuous signals of the intra-epidermal nerve fiber structural image is 200 nm or more, and the linear distance from one signal point to another signal point in a three-dimensional space ranges from 0 to 7.5 μm, and wherein the continuous signals connected together by at least three points constitute a string-like intra-epidermal nerve fiber structural signal segment, and the string-like intra-epidermal nerve fiber structural signal segment includes signal segments only existing in the epidermis, and the other signal segments extendedly crossing through the junctional layer of the skin epidermis and dermis to exist in the epidermis, and the shortest distance between the string-like intra-epidermal never fiber structural signal segments and the junctional layer of the epidermis and dermis is less than 20 μm, thereby constituting a plurality of nerve fibers;
calculating a total number of the plurality of nerve fibers of the total intra-epidermal nerve fiber in the acquisition area of the to-be-tested human skin and dividing it by a total acquisition area (mm²) of the captured images to obtain a density of the intra-epidermal nerve fiber of the to-be-tested human skin; and
evaluating levels of damage to the intra-epidermal nerve fiber of the to-be-tested human skin, and determining whether related neuropathy such as peripheral neuropathy is suffered, that is, the more severe the intra-epidermal nerve fiber is damaged, the lower the density is.

2. The method for non-invasive image-observing the density of an intra-epidermal nerve fiber of human skin of claim 1, wherein a normal value of the density of the intra-epidermal nerve fiber is from 50 to 60 (the number of the nerve fibers/mm²).

3. The method for non-invasive image-observing the density of an intra-epidermal nerve fiber of human skin of claim 1, wherein the intraepidermal nerve fiber is a C nerve fiber or A-delta nerve fiber, having a point-like structure.

4. The method for non-invasive image-observing the density of an intra-epidermal nerve fiber of human skin of claim 1, wherein the non-linear optical microscopy device is a third harmonic generation microscopy device (THG), a second harmonic generation microscopy device (SHG) plus a third harmonic generation microscopy device (THG), or a combination thereof, wherein the specification of the third harmonic generation microscopy device (THG) is NA≥0.75, the central wavelength: 1065-1450 nm, pulse width: <10 ps, and reverse collection of optical signals.

5. The method for non-invasive image-observing the density of an intra-epidermal nerve fiber of human skin of claim 1, wherein each nerve fiber extends along the same direction or a plurality of directions.

6. The method for non-invasive image-observing the density of an intra-epidermal nerve fiber of human skin of claim 1, wherein each nerve fiber is continuous in the same epidermis with no extension, or is continuous in different skin layers with extensions.

7. The method for non-invasive image-observing the density of an intra-epidermal nerve fiber of human skin of claim 1, wherein when each nerve fiber extends in different skin layers, it is a point-like signal and a point-like signal extension, or a point-like signal and a linear signal extension.

8. The method for non-invasive image-observing the density of an intra-epidermal nerve fiber of human skin of claim 1, wherein each nerve fiber is not arranged as a circle to eliminate cell signals in the skin.

9. The method for non-invasive image-observing the density of an intra-epidermal nerve fiber of human skin of claim 4, wherein the nonlinear optical microscopy device is a second harmonic generation microscopy device (SHG) plus a third harmonic generation microscopy device (THG) for observing the intra-epidermal nerve fiber structural image, and if the intra-epidermal nerve fiber extends in the horizontal direction (perpendicular to the forward direction of the laser light),
as is known through numerical simulation, it is used for generating a high-strength third harmonic generation, so that the intra-epidermal nerve fiber signal is observed in the same skin layer as linear and continuous;
and if the intra-epidermal nerve fiber extends in the vertical direction (parallel to the forward direction of the laser light), it is known through numerical simulation, that if it is a simple cylindrical thin line, the intensity of the third harmonic generation is extremely small, it must conform to the structure of the varicosity to generate high-strength third harmonic generation, so that the intra-epidermal nerve fiber signal is observed in different skin layers as a point-like and point-like extension, or a point-like and linear extension.

10. The method for non-invasive image-observing the density of an intra-epidermal nerve fiber of human skin of claim 1, which further comprises a displaying step for displaying the image and/or the density value of each nerve fiber.

11. The method for non-invasive image-observing the density of an intra-epidermal nerve fiber of human skin of claim 1, wherein when observing the intra-epidermal nerve fiber image, the number of cells in a basal layer of the epidermis capable of reflecting visibility of the image is further observed, and wherein the number of cells in the basal layer is used to correct the density of the entire segment of the intra-epidermal nerve fiber and the density of the intra-epidermal nerve fiber.

12. An apparatus for non-invasive image-observing the density of an intra-epidermal nerve fiber of human skin, which comprises:
a nonlinear optical microscopy device for capturing an intra-epidermal nerve fiber structural image of an acquisition area of a to-be-tested human skin to observe continuous signals of the intra-epidermal nerve fiber structural image, wherein the continuous signals of the intra-epidermal nerve fiber structural image include a string-like or a claw-like structural signal of a peripheral intra-epidermal nerve fiber formed by crossing through a junctional layer of the human skin epidermis and dermis and extending into the epidermis, and a string-like or a claw-like structural signal of the peripheral intra-epidermal nerve fiber only located in the epidermis, wherein the peripheral intra-epidermal nerve fiber is an unmyelinated fiber in the epidermis of the human skin, and wherein the nonlinear optical microscopy device includes: a laser source for emitting laser light with a pulsed laser, and an image processing member for processing image signals, in which the laser light is focused on the intra-epidermal nerve fiber of the acquisition area of the to-be-tested human skin by using the image processing member to obtain continuous signals of the intra-epidermal nerve fiber structural image with a length having at least three points and continuous, wherein the continuous signals of the intra-epidermal nerve fiber structural image are third harmonic generation nonlinear optical signals generated after being excited by the laser light, the size of the signal point of each of the continuous signals of the intra-epidermal nerve fiber structural image is 200 nm or more, and the linear distance from one signal point to another signal point in a three-dimensional space ranges from 0 to 7.5 μm, and wherein the continuous signals connected together by at least three points constitute a string-like intra-epidermal nerve fiber structural signal segment, and the string-like intra-epidermal nerve fiber structural signal segment includes signal segments only existing in the epidermis, and the other signal segments extendedly crossing through the junctional layer of the skin epidermis and dermis to exist in the epidermis, and the shortest distance between the string-like intra-epidermal never fiber structural signal segments and the junctional layer of the epidermis and dermis is less than 20 μm, thereby constituting a plurality of nerve fibers;
a calculation element for calculating a total number of the plurality of nerve fibers of the total intra-epidermal nerve fiber in the acquisition area of the to-be-tested human skin and dividing it by a total acquisition area (mm²) of the captured images to obtain a density of the intra-epidermal nerve fiber of the to-be-tested human skin; and
an evaluation and judging element for evaluating levels of damage to the intra-epidermal nerve fiber of the to-be-tested human skin, and determining whether related neuropathy such as peripheral neuropathy is suffered, that is, the more severe the intra-epidermal nerve fiber is damaged, the lower the density is.

13. The apparatus for non-invasive image-observing the density of an intra-epidermal nerve fiber of human skin of claim 12, which further comprises a displaying element for displaying the image and/or the density of each nerve fiber.

14. The apparatus for non-invasive image-observing the density of an intra-epidermal nerve fiber of human skin of claim 12, wherein a normal value of the density of the intra-epidermal nerve fiber is from 50 to 60 (the number of the nerve fibers/mm²).

15. The apparatus for non-invasive image-observing the density of an intra-epidermal nerve fiber of human skin of claim 12, wherein the intraepidermal nerve fiber is a C nerve fiber or A-delta nerve fiber, having a point-like structure.

16. The apparatus for non-invasive image-observing the density of an intra-epidermal nerve fiber of human skin of claim 12, wherein the non-linear optical microscopy device is a third harmonic generation microscopy device (THG), a second harmonic generation microscopy device (SHG) plus a third harmonic generation microscopy device (THG), or a combination thereof, wherein the specification of the third harmonic generation microscopy device (THG) is NA≥0.75, the central wavelength: 1065-1450 nm, pulse width: <10 ps, and reverse collection of optical signals.

17. The apparatus for non-invasive image-observing the density of an intra-epidermal nerve fiber of human skin of claim 12, wherein each nerve fiber extends along the same direction or a plurality of directions.

18. The apparatus for non-invasive image-observing the density of an intra-epidermal nerve fiber of human skin of claim 12, wherein each nerve fiber is continuous in the same epidermis with no extension, or is continuous in different skin layers with extensions.

19. The apparatus for non-invasive image-observing the density of an intra-epidermal nerve fiber of human skin of claim 12, wherein when each nerve fiber extends in different skin layers, it is a point-like signal and a point-like signal extension, or a point-like signal and a linear signal extension.

20. The apparatus for non-invasive image-observing the density of an intra-epidermal nerve fiber of human skin of claim 12, wherein each nerve fiber is not arranged as a circle to eliminate cell signals in the skin.

21. The apparatus for non-invasive image-observing the density of an intra-epidermal nerve fiber of human skin of claim 16, wherein the nonlinear optical microscopy device is a second harmonic generation microscopy device (SHG) plus a third harmonic generation microscopy device (THG) for observing the intra-epidermal nerve fiber structural image, and if the intra-epidermal nerve fiber extends in the horizontal direction (perpendicular to the forward direction of the laser light),
as is known through numerical simulation, it is used for generating a high-strength third harmonic generation, so that the intra-epidermal nerve fiber signal is observed in the same skin layer as linear and continuous;
and if the intra-epidermal nerve fiber extends in the vertical direction (parallel to the forward direction of the laser light), it is known through numerical simulation, that if it is a simple cylindrical thin line, the intensity of the third harmonic generation is extremely small, it must conform to the structure of the varicosity to generate high-strength third harmonic generation, so that the intra-epidermal nerve fiber signal is observed in different skin layers as a point-like and point-like extension, or a point-like and linear extension.

22. The apparatus for non-invasive image-observing the density of an intra-epidermal nerve fiber of human skin of claim 12, wherein when observing the intra-epidermal nerve fiber image, the number of cells in a basal layer of the epidermis capable of reflecting visibility of the image is further observed, and wherein a correction element is further provided for correcting the density of the intra-epidermal nerve fiber, via using the number of cells in the basal layer.
